(19) United States
(12) Patent Application Publication
(10) Pub. No.: US 2024/0065664 A1
(43) Pub. Date: Feb. 29, 2024

(54) PHYSIOLOGICAL SIGNAL MEASUREMENT DEVICE

(71) Applicant: Accurate Meditech Inc, New Taipei (TW)

(72) Inventors: CHENG YAN GUO, New Taipei (TW); KUAN JEN WANG, New Taipei (TW); PEI-MING CHIEN, New Taipei (TW); HAO-CHING CHANG, New Taipei (TW)

(21) Appl. No.: 17/894,404

(22) Filed: Aug. 24, 2022

(51) Int. Cl. A61B 7/04; A61B 5/00; A61B 5/02; A61B 5/024

(52) U.S. Cl. CPC A61B 7/04; A61B 5/02007; A61B 5/02438; A61B 5/6824; A61B 5/6831; A61B 2562/0204; A61B 2562/0233; A61B 2562/043; A61B 2562/063

(57) ABSTRACT

A physiological signal measurement device is disclosed. In some implementations, the physiological signal measurement device includes a fixing element, a rack, a first sensor, and a second sensor. The fixing element is configured to be fixed on a limb of a user. The rack is configured to engage the fixing element and includes a first end and a second end distal to the first end. The first sensor is disposed on the first end of the rack. The sensor is disposed on the second end of the rack. The first end of the rack has a first stiffness, the second end of the rack has a second stiffness, and the first stiffness is higher than the second stiffness.

### FIELD

The present disclosure generally relates to physiological signal measurement and, more specifically, to a structural design of physiological signal measurement devices that improves accuracy of the physiological signal measurement.

### BACKGROUND

For acquisition of physiological signals, at least three types of sensors are generally applied, namely, acoustic sensors, optical sensors, and electronic sensors. The acoustic sensors, such as stethoscopes and piezoelectric sensors, are used to acquire acoustic signals; the optical sensors, such as near infrared spectroscopy (NIRS) sensors, are used to acquire optical signals; and the electronic sensors, such as electrocardiography (ECG) sensors, are used to acquire electronic signals. According to different physiological signals, different sensors may be applied, and they may operate differently. For example, some sensors (e.g., NIRS sensors, etc.) may acquire the physiological signals remotely, and some sensors (e.g., piezoelectric sensors, ECG sensors, etc.) may need to attach onto a user's skin with good adhesion without sacrificing vibration response or conductivity.

Nevertheless, good adhesion to the user's skin may not be enough for purposes of accuracy in terms of acquisition of the user's pulse signals. Specifically, sensors may need to attach onto the user's skin for collecting vibrations resulting from the pulse. However, the vibrations may be absorbed by muscles and fats, thereby decrease the sensitivity, as well as the accuracy, of the sensor. As a result, the depth of the artery becomes an important factor when acquiring pulse signals.

### SUMMARY

The present disclosure is directed to physiological signal measurement devices which may take the depth of the artery into consideration and may possess an improved accuracy.

According to a first aspect of the present disclosure, a physiological signal measurement device includes a fixing element, a rack, a first sensor, and a second sensor. The fixing element is configured to fixed on a limb of a user. The rack is configured to engage the fixing element and includes a first end and a second end distal to the first end. The first sensor is disposed on the first end of the rack. The second sensor is disposed on the second end of the rack. The first end of the rack has a first stiffness, the second end of the rack has a second stiffness, and the first stiffness is higher than the second stiffness.

In an implementation of the first aspect of the present disclosure, each of the first sensor and the second sensor include an acoustic wave sensor.

In another implementation of the first aspect of the present disclosure, the first sensor is configured to detect a first vessel-related signal of the user at a first point on the limb, the second sensor is configured to detect a second vessel-related signal of the user at a second point on the limb, and the first point is proximal to the second point.

In another implementation of the first aspect of the present disclosure, the fixing element includes a band strap, and when the band strap forms a ring in a wearing state, a first perimeter of the ring is longer than a second perimeter of the ring.

In another implementation of the first aspect of the present disclosure, the first perimeter of the ring is on a same side as the first end of the rack, and the second perimeter of the ring is on a same side as the second end of the rack.

In another implementation of the first aspect of the present disclosure, the band strap has an arc shape.

In another implementation of the first aspect of the present disclosure, the fixing element includes a frame part and a fixing part, the frame part is configured to detachably engage the rack, and the fixing part is configured to secure the frame part to the limb of the user.

In another implementation of the first aspect of the present disclosure, the frame part of the fixing element includes a through-hole structure, and the through-hole structure corresponds to the first sensor and the second sensor when the rack engages the frame part of the fixing element.

In another implementation of the first aspect of the present disclosure, a thickness of the frame part gradually diminishes from a through-hole side to a fixing part side.

In another implementation of the first aspect of the present disclosure, the physiological signal measurement device further includes a host module including a controller and a first connection interface coupled to the controller. The rack further includes a second connection interface coupled to the first sensor and the second sensor, and the second connection interface is configured to connect to the first connection interface.

In another implementation of the first aspect of the present disclosure, the host module further includes a housing configured to accommodate the controller and that includes a latch. The latch is configured to detachably engage the rack.

In another implementation of the first aspect of the present disclosure, the physiological signal measurement device further includes a vibration collection structure corresponding to one of the first sensor or the second sensor. The vibration collection structure includes a rigid part and a rubber part covering the rigid part, the corresponding one of the first sensor or the second sensor is located at a center of the rigid part, and the rubber part is configured to contact skin of the user.

In another implementation of the first aspect of the present disclosure, the rigid part includes a cone-shaped structure configured to collect mechanical waves detectable by the corresponding one of the first sensor or the second sensor.

In another implementation of the first aspect of the present disclosure, the rubber part includes an innermost portion, at least one middle portion, and an outermost portion. The innermost portion and the outermost portion are in contact with the rigid part and the at least one middle portion is not in contact with the rigid part.

In another implementation of the first aspect of the present disclosure, the rubber part includes an innermost portion, at least one middle portion and an outermost portion. A thickness of the innermost portion and a thickness of the outermost portion are thicker than a thickness of the at least one middle portion. The at least one middle portion forms a cavity within the rubber part.

In another implementation of the first aspect of the present disclosure, a largest radius of the cavity is not larger than a largest radius of a cone-shaped structure of the rigid part.

In another implementation of the first aspect of the present disclosure, the physiological signal measurement device further includes a third sensor and a controller coupled to the first sensor, the second sensor, and the third sensor. The third sensor is disposed on the rack and positioned between the first sensor and the second sensor. The third sensor is configured to detect a vessel-related signal of the user. The first sensor, the second sensor, and the third sensor are arranged in a straight line on the rack. The controller is configured to: determine whether a pulse wave is detected by both of the first sensor and the second sensor; and in response to determining that the pulse wave is detected by both of the first sensor and the second sensor, actuate the third sensor or start to apply the vessel-related signal detected by the third sensor for calculating an output of the physiological signal measurement device.

In another implementation of the first aspect of the present disclosure, each of the first sensor and the second sensor includes an acoustic wave sensor, and the third sensor includes an optical sensor.

According to a second aspect of the present disclosure, a physiological signal measurement device includes a rack including a first end and a second end distal to the first end. The first end is configured to receive a first sensor, and the second end is configured to receive a second sensor. The first end of the rack includes a first U-shaped recessed structure, and the second end of the rack includes a second U-shaped recessed structure. Two parallel sides of the first U-shaped recessed structure are shorter than two parallel sides of the second U-shaped recessed structure.

According to a third aspect of the present disclosure, a physiological signal measurement device includes a rack including a first end and a second end distal to the first end. The first end is configured to receive a first sensor, and the second end is configured to receive a second sensor. The first end of the rack includes a first spring, and the second end of the rack includes a second spring. A spring constant of the first spring is larger than a spring constant of the second spring.

In light of the above-mentioned physiological signal measurement device, stiffnesses of two ends of the rack are different and as such deformations on the two ends of the rack may be different under a same pressure. Hence, two sensors disposed on the rack can be pressed into a user's skin at different depths when the user wearing the physiological signal measurement device on his/her limb. In such a design, a shape of the limbs (e.g., usually in a cone-shape) is taken into consideration such that the signal absorption by tissues (e.g., muscles, fats, etc.) of the user may be eliminated or compensated. Accordingly, the accuracy of the physiological signal measurement device can be improved.

### BRIEF DESCRIPTION OF THE DRAWINGS

Aspects of the present disclosure are best understood from the following detailed description when read with the accompanying figures. Various features are not drawn to scale. Dimensions of various features may be arbitrarily increased or reduced for clarity of discussion.

FIG. 1 is a perspective view of a physiological signal measurement device according to an example implementation of the present disclosure.

FIG. 2 is an exploded view of a physiological signal measurement device according to an example implementation of the present disclosure.

FIG. 3A is a perspective view of a sensor module according to an example implementation of the present disclosure.

FIG. 3B is a top view of a sensor module according to an example implementation of the present disclosure.

FIG. 3C is a bottom view of a sensor module according to an example implementation of the present disclosure.

FIG. 3D is a side sectional view of a sensor module according to an example implementation of the present disclosure.

FIG. 4A is a schematic diagram of a spring structure of an end of a rack of a sensor module according to an example implementation of the present disclosure.

FIG. 4B is a schematic diagram of a spring structure of an end of a rack of a sensor module according to another example implementation of the present disclosure.

FIG. 4C is a schematic diagram of a spring structure of an end of a rack of a sensor module according to yet another example implementation of the present disclosure.

FIG. 5A is a perspective view of a host module according to an example implementation of the present disclosure.

FIG. 5B is a front view of a host module according to an example implementation of the present disclosure.

FIG. 5C is a rear view of a host module according to an example implementation of the present disclosure.

FIG. 6A is a perspective view of a fixing element in a wearing state according to an example implementation of the present disclosure.

FIG. 6B is a top view of a fixing element in an extension state according to an example implementation of the present disclosure.

FIG. 6C is a side view of a fixing element in a wearing state according to an example implementation of the present disclosure.

FIG. 7 is a flowchart of a detection method performed by a physiological signal measurement device according to an example implementation of the present disclosure.

### DESCRIPTION

The following contains specific information pertaining to example implementations in the present disclosure. The drawings and their accompanying detailed disclosure are directed to merely example implementations of the present disclosure. However, the present disclosure is not limited to merely these example implementations. Other variations and implementations of the present disclosure will occur to those skilled in the art. Unless noted otherwise, like or corresponding elements among the figures may be indicated by like or corresponding reference numerals. Moreover, the drawings and illustrations in the present disclosure are generally not to scale and are not intended to correspond to actual relative dimensions.

For consistency and ease of understanding, like features are identified (although, in some examples, not illustrated) by numerals in the example figures. However, the features in different implementations may differ in other respects, and thus shall not be narrowly confined to what is illustrated in the figures.

References to “one implementation,” “an implementation,” “example implementation,” “various implementations,” “some implementations,” “implementations of the present disclosure,” etc., may indicate that the implementation(s) of the present disclosure may include a particular feature, structure, or characteristic, but not every possible implementation of the present disclosure necessarily includes the particular feature, structure, or characteristic. Further, repeated use of the phrase “in one implementation,” “in an example implementation,” or “an implementation,” do not necessarily refer to the same implementation, although they may. Moreover, any use of phrases like “implementations” in connection with “the present disclosure” are never meant to characterize that all implementations of the present disclosure must include the particular feature, structure, or characteristic, and should instead be understood to mean “at least some implementations of the present disclosure” include the stated particular feature, structure, or characteristic. The term “coupled” is defined as connected, whether directly or indirectly through intervening components, and is not necessarily limited to physical connections. The term “comprising,” when utilized, means “including but not necessarily limited to”; it specifically indicates open-ended inclusion or membership in the disclosed combination, group, series, and the equivalent.

The term “and/or” herein is only an association relationship for describing associated objects and represents that three relationships may exist; for example, A and/or B may represent that: A exists alone, A and B exist at the same time, and B exists alone. “A and/or B and/or C” may represent that at least one of A, B, and C exists. The character “/” used herein generally represents that the former and latter associated objects are in an “or” relationship.

Additionally, for a non-limiting explanation, specific details, such as functional entities, techniques, and the like, are set forth for providing an understanding of the disclosed technology. In other examples, detailed disclosure of well-known methods, technologies, systems, architectures, and the like are omitted so as not to obscure the present disclosure with unnecessary details.

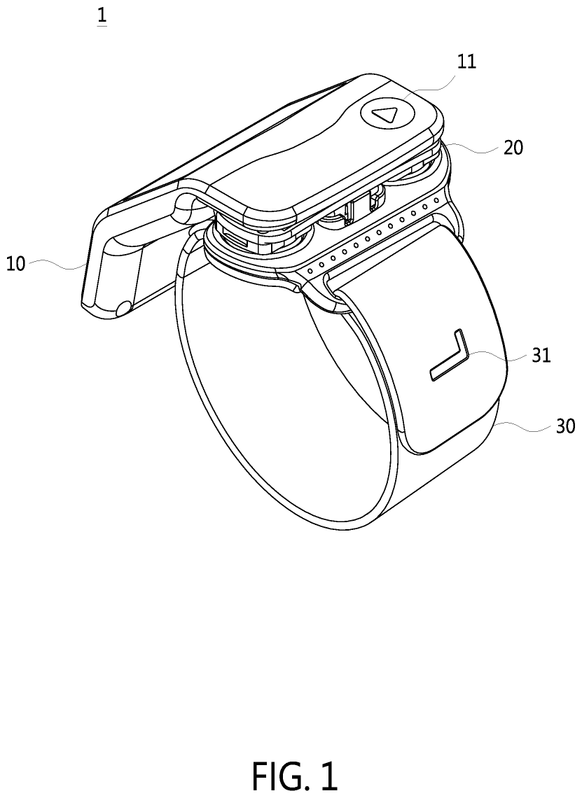

FIG. 1 is a perspective view of a physiological signal measurement device according to an example implementation of the present disclosure.

Referring to FIG. 1, in some implementations, a physiological signal measurement device 1 may include a host module 10, a sensor module 20, and a fixing element 30. The host module 10 may be connected to the sensor module 20, and the sensor module 20 may be engaged with the fixing element 30. The fixing element 30 is configured to be fixed on a limb (e.g., a wrist) of a user. Therefore, the user may wear the physiological signal measurement device 1 by using the fixing element 30, the sensor module 20 may detect physiological signals of the user, and the host module 10 may generate an output based on the physiological signals detected by the sensor module 20.

In some implementations, the host module 10, the sensor module 20, and the fixing element 30 may be three separate modules. That is, the host module 10 may be detachably connected to the sensor module 20, and the sensor module 20 may be detachably engaged with the fixing element 30. Therefore, the user may first tie the fixing element 30 on a limb (e.g., a wrist) of the user, and then engage the sensor module 20 and the host module 10 with the fixing element 30, which is already fixed on the limb of the user.

In some implementations, the host module 10 may include a direction mark 11 for indicating a direction associated with the host module 10 when connecting the host module 10 with the sensor module 20. In some implementations, the fixing element 30 may include a left mark 31 or a right mark (not shown) for indicating which wrist should be tied by the fixing element 30.

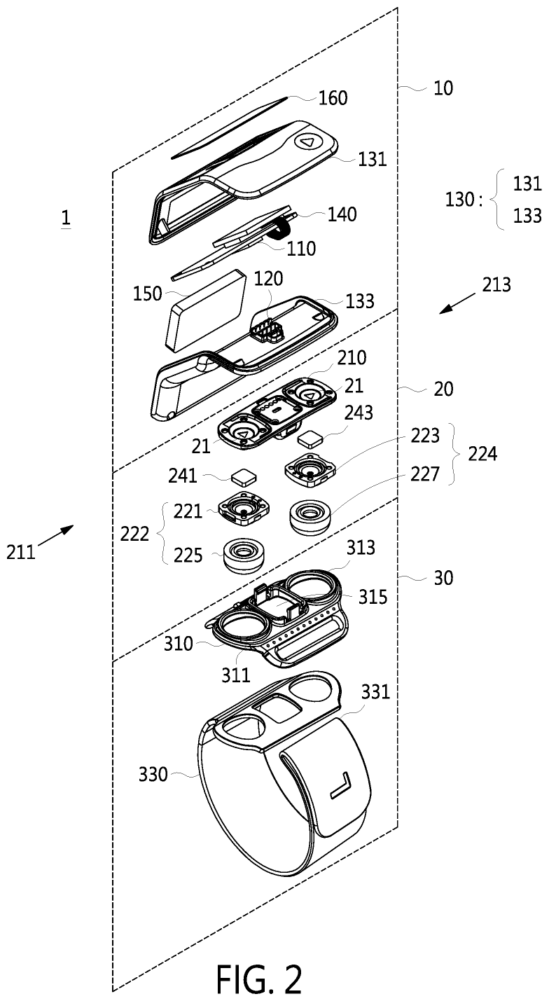

FIG. 2 is an exploded view of a physiological signal measurement device according to an example implementation of the present disclosure.

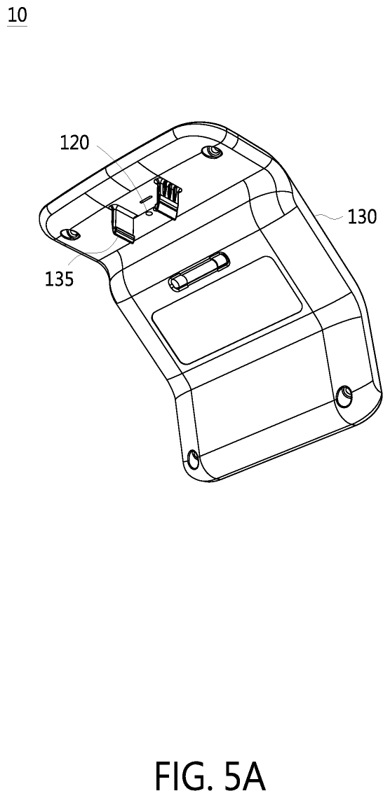

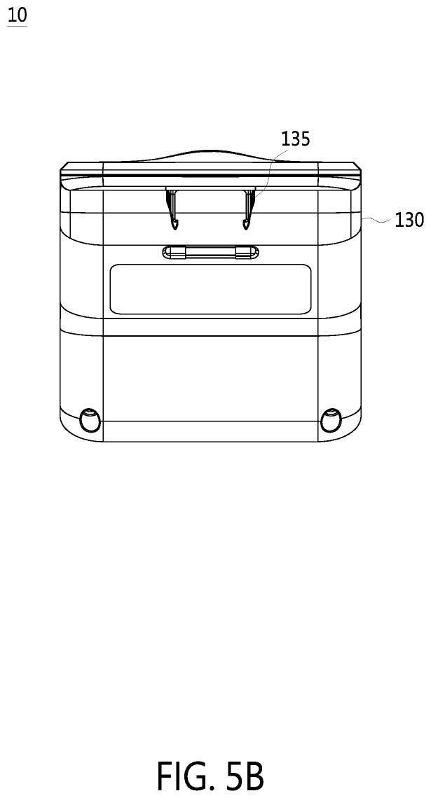

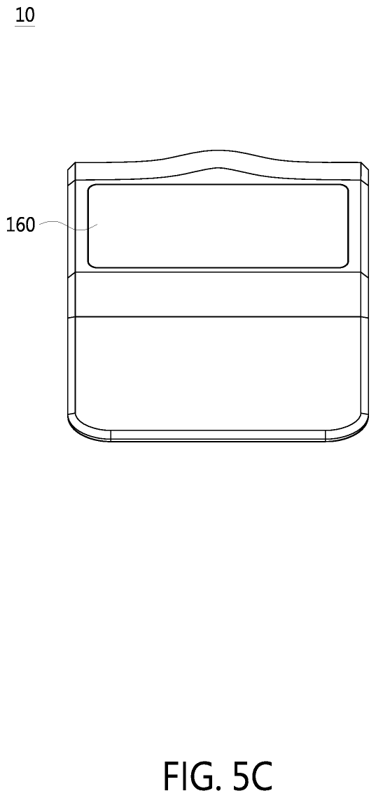

Referring to FIG. 2, in some implementations, the host module 10 may include a controller 110, a first connection interface 120, and a housing 130. The housing 130 is configured to accommodate the controller 110. The controller 110 is coupled to the first connection interface 120, and the first connection interface 120 is configured to communicate with another module (e.g., the sensor module 20). In some implementations, the housing 130 further includes a latch 135 (not shown in FIG. 2, but instead depicted in FIGS. 5A and 5B) for detachably engaging with the other module (e.g., the sensor module 20).

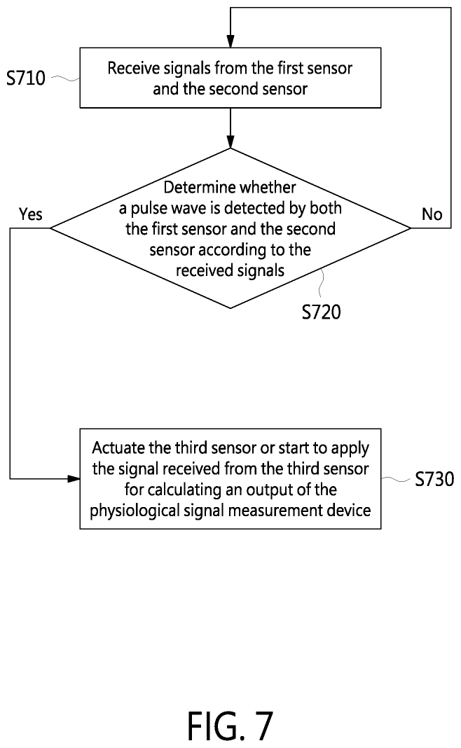

In some implementations, the controller 110 may be, for example, a central processing unit (CPU) or another programmable general-purpose or special-purpose microprocessor, a digital signal processor (DSP), a programmable controller, an application-specific integrated circuit (ASIC), a programmable logic device (PLD), or other similar components or a combination of the components. In addition, the host module may include a memory (not depicted in FIG. 2) storing computer-executable instructions that, when executed by the controller 110, cause the physiological signal measurement device 1 to perform various operations described herein, such as the detection method described below in conjunction with FIG. 7.

In some implementations, the housing 130 may further include an upper case 131 and a lower case 133, where the latch 135 may be disposed on the lower case 133 (e.g., on a downward-facing surface of the lower case 133).

In some implementations, the host module 10 may further include a printed circuit board (PCB) 140, a battery 150, and an operation panel 160, where the controller 110 may be disposed on the PCB 140 and further coupled to the operation panel 160. The operation panel 160 may be, for example, a touch panel or a display panel with several operation buttons, and configured to receive an external command from the user and display output information of the physiological signal measurement device 1. The battery 150 may be configured to provide power to the physiological signal measurement device 1.

It should be noted that one of ordinary skill in the art may equip the host module 10 with other functionalities or components according to need, which is not limited in the present disclosure.

Referring to FIG. 2 again, in some implementations, the sensor module 20 may include a first sensor 241, a second sensor 243, and a rack 210. The rack 210 may, for example, include a first end 211 and a second end 213. The first sensor 241 is disposed at the first end 211 of the rack 210, and the second sensor 243 is disposed at the second end 213 of the rack 210.

In some implementations, the first end 211 and the second end 213 of the rack 210 may be formed by different structures or materials to have different tensile strengths, such that the stiffness of the first end 211 of the rack 210 may be higher than the stiffness of the second end 213 of the rack 210. That is, under a same pressure or external force, the deformation of the second end 213 of the rack 210 may be larger than that of the first end 211 of the rack 210.

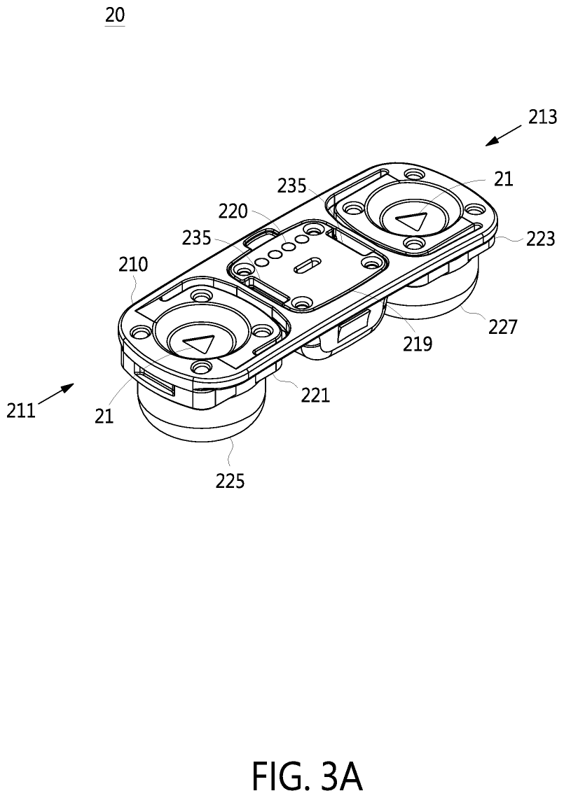

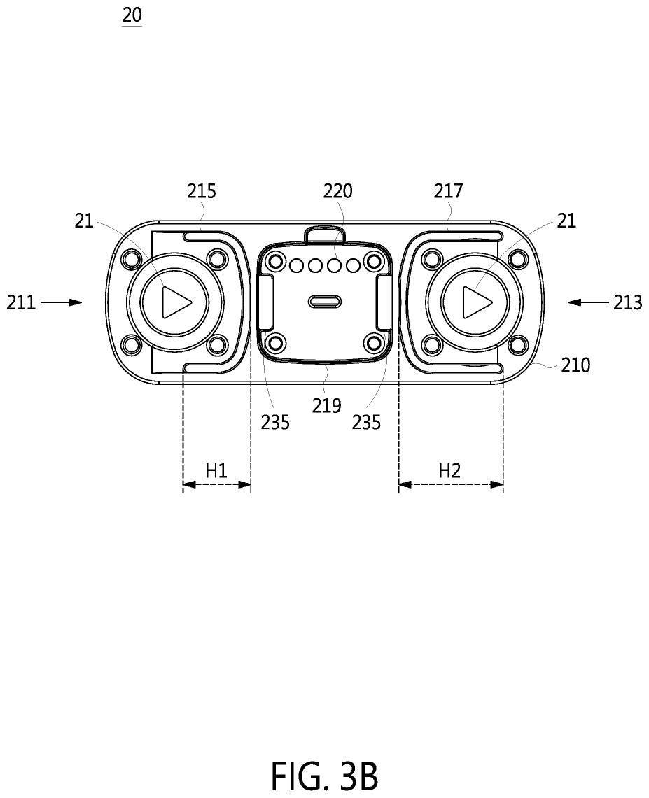

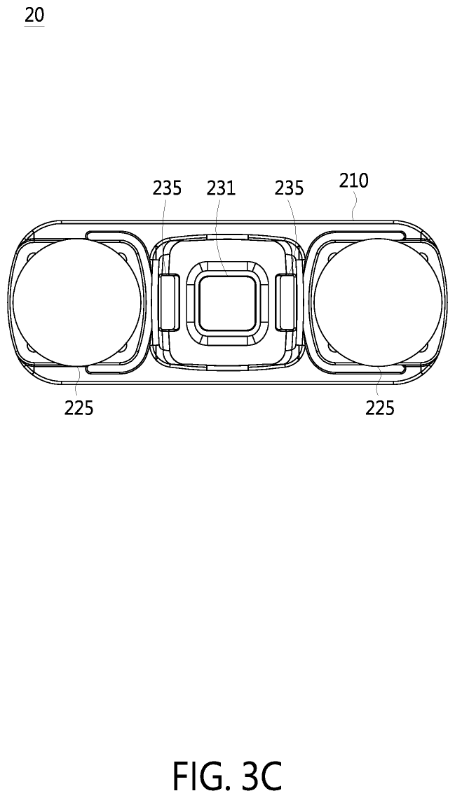

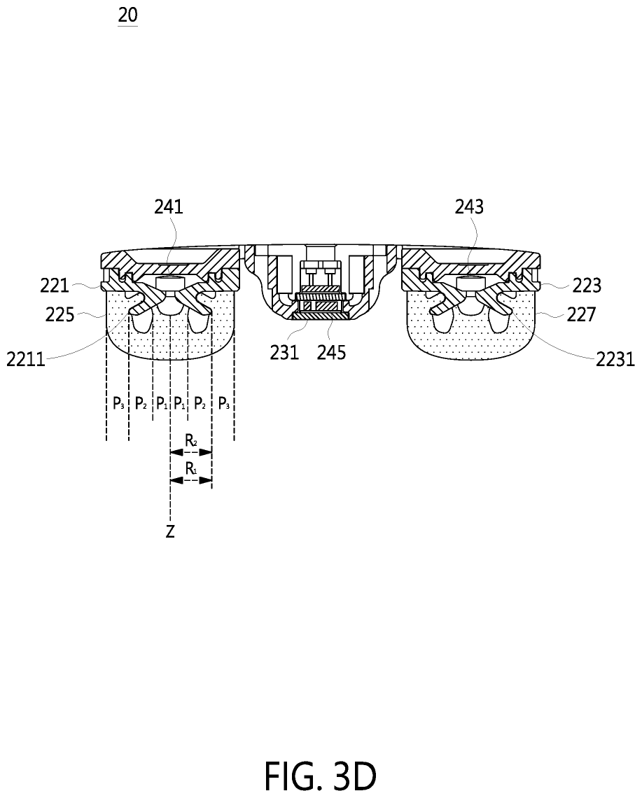

In some implementations, the rack 210 may further include a notch 235 (not shown in FIG. 2, but illustrated in FIGS. 3A, 3B, and 3C). The notch 235 corresponds to the latch 135 of the host module 10 such that the host module 10 may be detachably engaged with the sensor module 20 through the latch 135 and the notch 235. In some implementations, the sensor module 20 may further include a second connection interface 220 (not shown in FIG. 2, but instead illustrated in FIGS. 3A and 3B) coupled to all sensors (e.g., the first sensor 241 and the second sensor 243) in the sensor module 20. In a case that the host module 10 is engaged with the sensor module 20 (e.g., through the latch 135 and the notch 235), the first connection interface 120 of the host module 10 may be electrically connected to the second connection interface 220 of the sensor module 20, such that the controller 110 of the host module 10 may acquire signals from sensors of the sensor module 20 and/or the battery 150 may provide power to the sensor module 20.

In some implementations, the sensor module 20 may further include a third sensor 245 (not shown in FIG. 2, but instead depicted in FIG. 3D). The detection target of the first sensor 241, the second sensor 243, and the third sensor 245 may be the same, but the type of the first sensor 241, the second sensor 243, and the third sensor 245 may be different. For example, the detection target of the first sensor 241, the second sensor 243, and the third sensor 245 may be an artery of the user, where the first sensor 241 and the second sensor 243 may be acoustic wave sensors and the third sensor 245 may be an optical sensor. In some implementations, the rack 210 may further include a cover 219 (not shown in FIG. 2, but instead illustrated in FIGS. 3A and 3B) for covering the third sensor 245. However, the present disclosure is not limited in the aforementioned example.

In some implementations, from a top view of the sensor module 20, the first sensor 241, the second sensor 243, and the third sensor 245 may be arranged in a straight line, where the third sensor 245 is located between the first sensor 241 and the second sensor 243. In such an arrangement, if the first sensor 241, the second sensor 243, and the third sensor 245 have a same detection target (e.g., an artery of the user), the first sensor 241 and the second sensor 243 can be in charge of positioning the detection target to ensure the third sensor 245 is correctly located.

In some implementations, the sensor module 20 may further include a first vibration collection structure 222 corresponding to the first sensor 241, and a second vibration collection structure 224 corresponding to the second sensor 243. Details of the vibration collection structures 222, 224 will be described below.

In some implementations, the sensor module 20 may further include at least one direction mark 21 for indicating a direction associated with the sensor module 20 when the user engages the sensor module 20 with the fixing element 30.

It should be noted that one of ordinary skill in the art may equip the sensor module 20 with other functionalities or components according to need, which is not limited in the present disclosure.

Referring to FIG. 2 again, in some implementations, the fixing element 30 may include a frame part 310 and a fixing part 330, where the frame part 310 is configured to detachably engage with the rack 210 of the sensor module 20 and the fixing part 330 is configured to fix on the user's limb.

In some implementations, the frame part 310 may include a through-hole structure (e.g., including three through-holes 311, 313, 315) corresponding to at least the first sensor 241 and the second sensor 243 of the sensor module 20 when the rack 210 engages the frame part 310 of the fixing element 30. Specifically, in a case that the rack 210 engages the frame part 310 of the fixing element 30, the first sensor 241 and the second sensor 243 may acquire physiological signals (e.g., vessel-related signals, such as pulse wave signals) through the through-hole structure (e.g., through-holes 311, 313, 315).

Advantageously, the user may wear the fixing element 30 on his/her limb and find the detection target (e.g., artery) by hand (e.g., feel the pulse wave) or by other instruments through the through-hole structure (e.g., through-holes 311, 313, 315) in advance. For example, the user may adjust the angle of the fixing element 30 by aligning the through-hole structure (e.g., through-holes 311, 313, 315) with the artery to make sure the first sensor 241 and the second sensor 243 would be correctly located when the sensor module 20 is engaged with the fixing element 30.

In some implementations, the through-hole structure (e.g., through-holes 311, 313, 315) may include multiple (e.g., three) through-holes each corresponding to a sensor. However, as long as the through-hole structure (e.g., through-holes 311, 313, 315) corresponds to the first sensor 241 and the second sensor 243 when the rack 210 engages the frame part 310 of the fixing element 30, numbers and shapes of the through-holes in the through-hole structure are not limited in the present disclosure.

In some implementations, the fixing part 330 may be a band strap (e.g., including velcro 331) for wrapping about a limb of the user (e.g., to secure or affix the frame part 310 to the limb).

It should be noted that one of ordinary skill in the art may equip the fixing element 30 with other functionalities or components according to need, which is not limited in the present disclosure.

Details of each of the host module 10, the sensor module 20, and the fixing element 30 are described below.

FIG. 3A is a perspective view of the sensor module 20 according to an example implementation of the present disclosure; FIG. 3B is a top view of the sensor module 20 according to an example implementation of the present disclosure; FIG. 3C is a bottom view of the sensor module 20 according to an example implementation of the present disclosure; FIG. 3D is a side sectional view of the sensor module 20 according to an example implementation of the present disclosure.

Referring to FIG. 3A through FIG. 3D, in some implementations, the sensor module 20 may include the rack 210, the second connection interface 220, the first sensor 241, the second sensor 243, the third sensor 245, the first vibration collection structure 222 corresponding to the first sensor 241, and the second vibration collection structure 224 corresponding to the second sensor 243.

In some implementations, the rack 210 may form a space for accommodating the third sensor 245, and further include the cover 219 for covering the third sensor 245 and a window 231 (see FIGS. 3C and 3D) for exposing the third sensor 245. For example, the third sensor 245 may be an optical sensor (e.g., near infrared spectroscopy, or NIRS, sensor) that includes a light emission part and a light receiving part. Light emitted from the light emission part and light reflected toward the light receiving part may pass through the window 231.

In some implementations, the first vibration collection structure 222 may include a rigid part 221 and a rubber part 225 covering (e.g., capping over) the rigid part 221, as shown in FIG. 3D.

The rigid part 221 may be, for example, made from hard plastic (e.g., ABS or polycarbonate) and may include a cone-shape structure 2211. The first sensor 241 may be located at the center of the cone-shape structure 2211, and the cone-shape structure 2211 may work as a stethoscope which collects mechanical waves or sound waves received from the rubber part 225.

The rubber part 225 may be, for example, made from rubber and configured to contact skin of the user. In order to maintain the shape (e.g., a dome shape, as shown in FIG. 3D) of the rubber part 225 and thus avoid collapse of the rubber part 225 so that the rubber part 225 may more effectively conform to the skin surface, the rubber part 225 may be designed with different thicknesses (e.g., along the direction of an axis Z) from the center to the edge. Specifically, the rubber part 225 may include at least three portions P1 to P3 with different thicknesses from the center to the edge. The innermost portion P1 and the outermost portion P3 may be thicker than at least one middle portion P2, and may be in contact with the rigid part 221 for support. Instead of being in contact with the rigid part 221, the at least one middle portion P2 may form an annular cavity within the first vibration collection structure 222 for transmission of the mechanical waves. In some implementations, each of the at least three portions P1, P2, and P3 are rotationally symmetrical about the axis Z passing through the center of the rubber part 225.

It is noted that, for better collection of the mechanical waves, the largest radius R2 of the annular cavity formed by the at least one middle portion P2 may not be larger than the largest radius R1 of the cone-shape structure 2211 of the rigid part 221.

In some implementations, the second vibration collection structure 224 may include a rigid part 223 and a rubber part 227. The second vibration collection structure 224 may be similar to the first vibration collection structure 222, and therefore details of the second vibration collection structure are not repeated herein.

It should be noted that a depth of an artery on a limb usually gets shallower from a trunk portion (e.g., upstream) to an extremity portion (e.g., downstream) of the artery. In other words, the closer the arteries are to the extremities, the closer the arteries are to the skin surface. Therefore, it may be beneficial for sensor accuracy to press a sensor closer to the trunk portion deeper than a sensor closer to the extremity portion of the artery.

In light of the foregoing, in some implementations, a first end 211 of the rack 210 may have a higher stiffness than a second end 213 of the rack 210. If the sensor module 20 is fixed on a limb of the user by the fixing element 30 and the fixing part 33 (e.g., the band strap) applies approximately equal downward force to both ends 211, 213 of the rack 210, since the first end 211 of the rack 210 has a relatively high stiffness, it may undergo a smaller amount of deformation than the second end 213, thus being pressed deeper into the user's skin than the second end 213 under equal downward force (e.g., applied by the fixing element 30). Therefore, the first sensor 241 disposed on the first end 211 of the rack 210 may be configured to detect a first vessel-related signal of the user at a first point closer to the user's trunk. On the other hand, the second end 213 of the rack has a relatively low stiffness, and thus may exhibit a larger amount of deformation than the first end 211, thus being pressed shallower into the user's skin than the first end 211 under equal downward force (e.g., applied by the fixing element 30). Therefore, the second sensor 243 disposed on the second end 213 of the rack 210 may be configured to detect a second vessel-related signal of the user at a second point distal to the user's trunk. As a result, the signal absorption by tissues (e.g., muscles, fats, etc.) of the user may be eliminated or compensated for, and thus the accuracy of the two sensors 241 and 243 may be improved.

In some implementations, the downward-force difference or the stiffness difference of the first end 211 and the second end 213 of the rack 210 may be achieved by forming different sizes of U-shaped recessed structures on the first end 211 and the second end 213 of the rack 210.

For example, referring to FIG. 3B, the rack 210 may include a first U-shaped recessed structure 215 on the first end 211, and a second U-shaped recessed structure 217 on the second end 213. The lengths of two parallel sides of the U-shaped recessed structures 215, 217 may be negatively related to the stiffness. In other words, the longer the parallel portion of the U-shaped recessed structure 215, 217 is, the lower stiffness is on the corresponding end of the rack 210. Tn the present example, a length H1 of the two parallel sides of the first U-shaped recessed structure 215 is shorter than a length H2 of the two parallel sides of the second U-shaped recessed structure 217. Therefore, stiffness of the first end 211 of the rack 210 is higher than that of the second end 213 of the rack 210.

In some implementations, the downward-force difference or the stiffness difference of the first end 211 and the second end 213 of the rack 210 may be achieved by including springs with different spring constants for the first end 211 and the second end 213 of the rack 210. Several examples are described below.

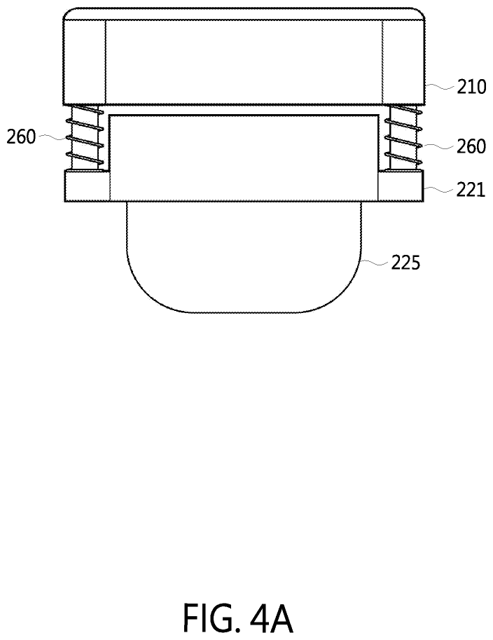

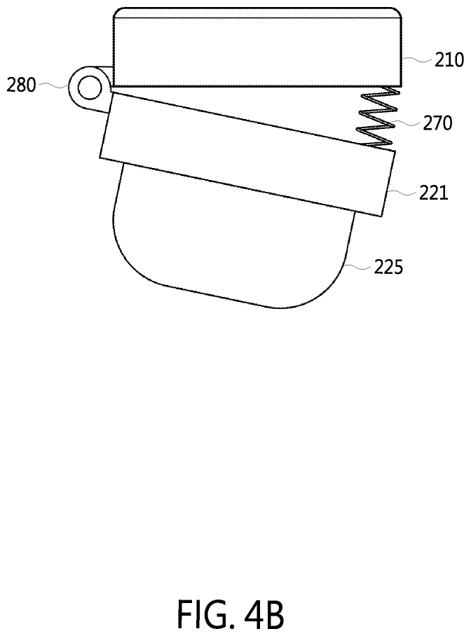

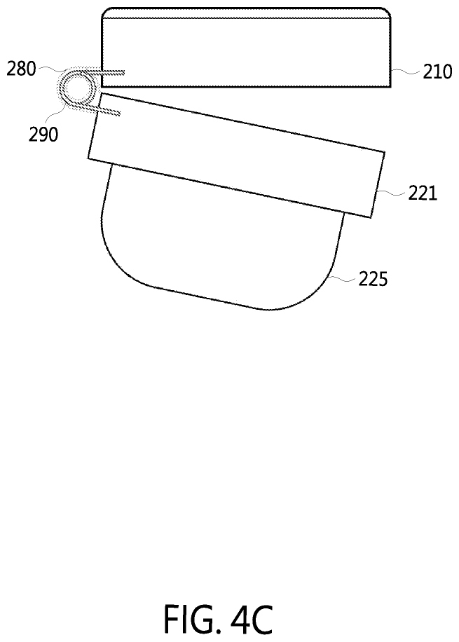

FIG. 4A is a schematic diagram of a spring structure of an end (e.g., the first end 211) of the rack 210 of the sensor module 20 according to an example implementation of the present disclosure.

Referring to FIG. 4A, at least one spring 260 may be installed on the first end 211 of the rack 210, may surround and/or be coupled to the rigid part 221, and may be configured to provide resistance (or assistance) when a downward force is applied to the rigid part 221, as well as the rubber part 225 and the first sensor 241 located in the rigid part 221, via the rack 210. On the other side not shown in FIG. 4A, at least one other spring may be installed on the second end 213 of the rack 210, may surround and/or be coupled to the rigid part 223, and may be configured to provide resistance (or assistance) when a downward force is applied to the rigid part 223, as well as the rubber part 227 and the second sensor 243 located in the rigid part 223, via the rack 210.

In some implementations, in a case that the spring constant of the spring 260 is larger than that of the other spring, when the two ends 211, 213 of the rack 210 are pulled downward by the fixing element 30, the restoring force provided by the spring 260 of the first end 211 of the rack 210 is larger than the restoring force provided by the other spring of the second end 213 of the rack 210. Therefore, the first end 211 having the spring 260 with the larger spring constant is harder to deform than the second end 213 having the other spring with the smaller spring constant. Consequently, when the two ends 211, 213 of the rack 210 are pressed into a user's skin, the stiffness of the first end 211 of the rack 210, as provided by the spring 260, is higher than that of the second end 213 of the rack 210, as provided by the other spring.

However, the design of the springs to cause the two ends 211, 213 of the rack 210 to have different stiffnesses is not limited in the present disclosure. In other implementations, the springs may be designed such that an end of a rack having a spring with a smaller spring constant is stiffer than another end of the rack having another spring with a larger spring constant when the rack 210 is pressed into a user's skin.

FIG. 4B is a schematic diagram of a spring structure of an end (e.g., the first end 211) of the rack 210 of the sensor module 20 according to another example implementation of the present disclosure.

Referring to FIG. 4B, the rack 210 may further include two hinges 280 for connecting the ends 211, 213 to the rigid parts 221, 223. A spring 270 may be installed on the edge of the first end 211 of the rack 210 and may be configured to provide resistance (or assistance) when a downward force is applied to the rigid part 221, as well as the rubber part 225 and the first sensor 241 located in the rigid part 221, via the rack 210. On the other side not shown in FIG. 4B, another spring may be installed on the edge of the second end 213 of the rack 210 and may be configured to provide resistance (or assistance) when a downward force is applied to the rigid part 223, as well as the rubber part 227 and the second sensor 243 located in the rigid part 223, via the rack 210.

In some implementations, in a case that the spring constant of the spring 270 is larger than that of the other spring, when the two ends 211, 213 of the rack 210 are pulled downward by the fixing element 30, the restoring force provided by the spring 270 of the first end 211 of the rack 210 is larger than the restoring force provided by the other spring of the second end 213 of the rack 210. Therefore, the first end 211 having the spring 270 with the larger spring constant is harder to deform than the second end 213 having the other spring with the smaller spring constant. Consequently, when the two ends 211, 213 of the rack 210 are pressed into a user's skin, the stiffness of the first end 211 of the rack 210, as provided by the spring 270, is higher than that of the second end 213 of the rack 210, as provided by the other spring.

However, the design of the springs to cause the two ends 211, 213 of the rack 210 to have different stiffnesses is not limited in the present disclosure. In other implementations, the springs may be designed such that an end of a rack having a spring with a smaller spring constant is stiffer than another end of the rack having another spring with a larger spring constant when the rack 210 is pressed into a user's skin.

FIG. 4C is a schematic diagram of a spring structure of an end (e.g., the first end 211) of the rack 210 of the sensor module 20 according to yet another example implementation of the present disclosure.

Referring to FIG. 4C, the rack 210 may further include two hinges 280 for connecting the ends 211, 213 to the rigid parts 221, 223. A spring 290 and another spring may be installed at the two hinges 280, respectively. The springs installed at the hinges 280 may work as grips and configured to provide resistance (or assistance) when downward forces are applied to the rigid parts 221, 223, as well as the rubber parts 225, 227 and the sensors 241, 243 located in the rigid parts 221, 223, via the rack 210.

In some implementations, in a case that the spring constant of the spring 290 is larger than that of the other spring, when the two ends 211, 213 of the rack 210 are pulled downward by the fixing element 30, the restoring force provided by the spring 290 of the first end 211 of the rack 210 is larger than the restoring force provided by the other spring of the second end 213 of the rack 210. Therefore, the first end 211 having the spring 290 with the larger spring constant is harder to deform than the second end 213 having the other spring with the smaller spring constant. Consequently, when the two ends 211, 213 of the rack 210 are pressed into a user's skin, the stiffness of the first end 211 of the rack 210, as provided by the spring 290, is higher than that of the second end 213 of the rack 210, as provided by the other spring.

However, the design of the springs to cause the two ends 211, 213 of the rack 210 to have different stiffnesses is not limited in the present disclosure. In other implementations, the springs may be designed such that an end of a rack having a spring with a smaller spring constant is stiffer than another end of the rack having another spring with a larger spring constant when the rack 210 is pressed into a user's skin.

Although several examples are described above in conjunction with the drawings, it should be noted that detailed implementations of the stiffness difference of the two ends 211, 213 of the rack 210 are not limited in the present disclosure. One of ordinary skill in the art may adopt other designs to apply different downforces to the first sensor 241 and the second sensor 243.

FIG. 5A is a perspective view of the host module 10 according to an example implementation of the present disclosure; FIG. 5B is a front view of the host module 10 according to an example implementation of the present disclosure; FIG. 5C is a rear view of the host module 10 according to an example implementation of the present disclosure.

Referring to FIG. 5A, FIG. 5B, and FIG. 5C, in some implementations, several components (e.g., controller 110, printed circuit board 140, battery 150, memory, and operation panel 160, etc.) of the host module 10 may be packaged in the housing 130. At the lower case 133 of the housing 130, a latch 135 may be disposed thereon for engaging with the notch 235 of the sensor module 20. In addition, a first connection interface 120 corresponding to the second connection interface 220 may also be exposed to the lower case 133 of the housing 130. When the latch 135 is engaged with the notch 235 of the sensor module 20, the first connection interface 120 is electrically connected to the second connection interface 220 of the sensor module 20.

In some implementations, an operation panel 160 may be exposed through the upper case 131 of the housing 130 and configured to receive an external command from the user and display output information of the physiological signal measurement device 1. In some implementations, the controller 110 may receive signals from sensors of the sensor module 20, calculate the output information of the physiological signal measurement device 1, and display the output information through the operation panel 160. For example, the first sensor 241 and the second sensor 243 may be acoustic wave sensors (e.g., piezoelectric sensors) and configured to detect pulse waves of the user, and the third sensor 245 may be an optical sensor (e.g., a NIRS sensor) and configured to detect a photoplethysmogram intensity ratio (PIR). The controller 110 may calculate blood-related output information, such as blood pressure, according to the detected pulse wave, the PIR, and other data stored in the memory. However, details of the calculation are not limited in the present disclosure, and one of ordinary skill in the art may perform the calculations based on their knowledge or related documentation.

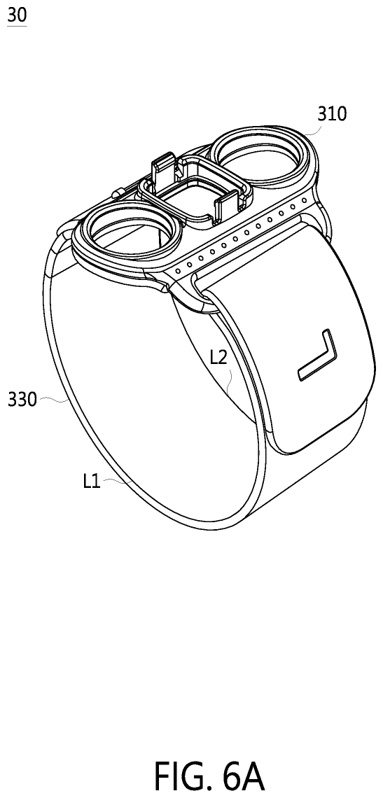

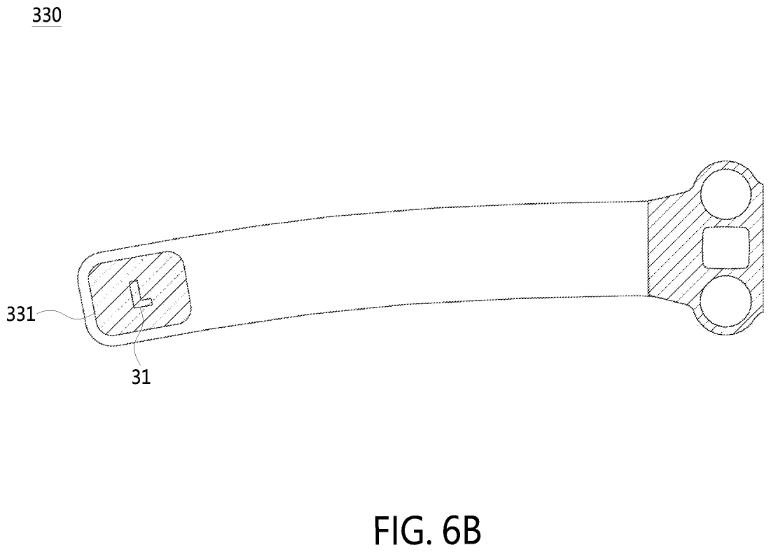

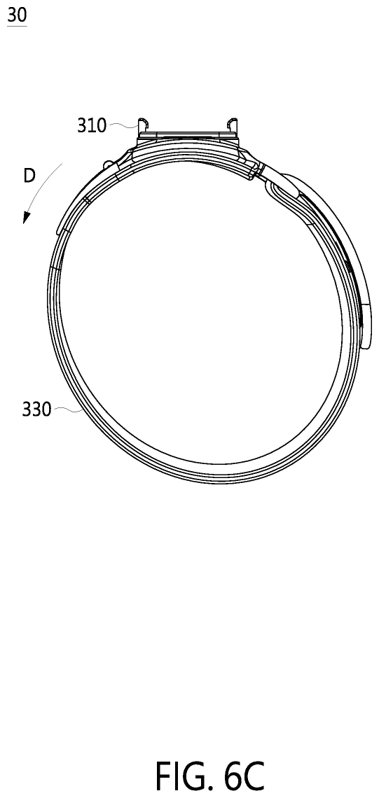

FIG. 6A is a perspective view of a fixing element in a wearing state according to an example implementation of the present disclosure; FIG. 6B is a top view of a fixing element in an extension state according to an example implementation of the present disclosure; FIG. 6C is a side view of a fixing element in a wearing state according to an example implementation of the present disclosure.

Referring to FIG. 6A, FIG. 6B, and FIG. 6C, in some implementations, the fixing element 30 may include the frame part 310 and the fixing part 330. For example, the frame part 310 may be made from elastic plastic materials such as Polypropylene or Nylon, and the fixing part 330 may be a band strap 330 made from laminated elastic fabrics with multiple layers such as breathable micro-fiber fabric on elastic micro-Velcro fabric with foam sheet insertion assembled with heated compression process.

In some implementations, a thickness of the frame part 310 may gradually diminish from a through-hole side to a fixing part side (e.g., in direction D shown in FIG. 6C). As such, the wearing comfort of the physiological signal measurement device 1 may be improved.

It should be noted that limbs of the user are usually cone-shaped or tapered instead of uniform cylinders. Accordingly, in some implementations, the fixing part 330 may be designed such that when the fixing part 330 forms a ring in a wearing state as shown in FIG. 6A, a first perimeter L1 of the ring is longer than a second perimeter L2 of the ring. In such a design, better conformity with a user's skin may be achieved and approximately equal downforces may be applied to both ends 211, 213 of the rack 210 by the fixing element 30 when the physiological signal measurement device 1 is worn on a limb of the user.

In some implementations, the first perimeter L1 may be, for example, on the same side as the first end 211 of the rack 210, and the second perimeter L2 may be, for example, on the same side as the second end 213 of the rack 210. Therefore, when the physiological signal measurement device 1 is worn on a limb of the user by the fixing part 330, the first perimeter L1 is proximal to (e.g., closer to the trunk of the user than) the second perimeter L2 and, as such, better conformity with a user's skin can be achieved and approximately equal downforces may be applied to both ends 211, 213 of the rack 210 by the fixing element 30.

In some implementations, the fixing part 330 may be a band strap, and the band strap may be in an arc shape, as shown in FIG. 6B. As a result, when the fixing part 330 forms the ring in the wearing state, the first perimeter L1 may be shorter than the second perimeter L2. The arc shape may be, for example, represented by an ellipse equation. However, it is noted that the present disclosure does not limit the arc shape of the band strap. In some implementations, the arc shape may be represented by a parabolic equation or a circular equation.

FIG. 7 is a flowchart of a detection method performed by a physiological signal measurement device according to an example implementation of the present disclosure.

In some implementations, the sensor module 20 may include the first sensor 241, the second sensor 243, and the third sensor 245. The first sensor 241 and the second sensor 243 may be a first type sensor for detecting a pulse wave and the third sensor 245 may be a second type sensor for detecting a vessel-related signal. For example, the first sensor 241 and the second sensor 243 may be acoustic wave sensors (e.g., piezoelectric sensors) and configured to detect a pulse wave of the user, and the third sensor 245 may be an optical sensor (e.g., a NIRS sensor) and configured to detect another vessel-related signal (e.g., PIR) of the user. From a top view of the sensor module 20, the first sensor 241, the second sensor 243, and the third sensor 245 are arranged in a straight line, and the third sensor 245 is located between the first sensor 241 and the second sensor 243. The controller 110 of the host module 10 may be coupled to the first sensor 241, the second sensor 243, and the third sensor 245 (e.g., through the first connection interface 120 and the second connection interface 220) and configured to perform the following detection method.

Referring to FIG. 7, in action S710, the controller 110 may receive signals from the first sensor 241 and the second sensor 243; in action S720, the controller 110 may determine whether a pulse wave is detected by both the first sensor 241 and the second sensor 243 according to the received signals. In a case of the controller 110 determining that the pulse wave is detected by both the first sensor 241 and the second sensor 243 according to the received signals, the process goes to action S730, where the controller 110 may actuate the third sensor 245 or may start to apply the signal received from the third sensor 245 for calculating an output of the physiological signal measurement device. In a case of the controller 110 determining that the pulse wave is not detected by both the first sensor 241 and the second sensor 243 according to the received signals, the process may return to action S710.

In some implementations, a threshold may be preset for the first sensor 241 and the second sensor 243. If an amplitude of a signal from a sensor (e.g., the first sensor 241 or second sensor 243) reaches the threshold, the controller 110 may determine that the sensor detects the pulse wave, which means that the sensor is correctly located on an artery of the user. In some implementations, in a case of the controller 110 determining that the pulse wave is detected by both the first sensor 241 and the second sensor 243 according to the received signals, the controller 110 may send a message (e.g., through the operation panel 160) indicating that the sensor module 20 is correctly located.

Specifically, as the first sensor 241, the second sensor 243, and the third sensor 245 are arranged in a straight line, the third sensor 245 should be correctly located on the artery if the pulse wave is detected by both the first sensor 241 and the second sensor 243. Therefore, the controller 110 may actuate the third sensor 245 or may start to apply the signal received from the third sensor 245 for calculating an output of the physiological signal measurement device only when the pulse wave is detected by both the first sensor 241 and the second sensor 243. As such, accuracy of the signal from the third sensor 245 may be improved.

In some implementations, in a case of the controller 110 determining that the pulse wave is not detected by both the first sensor 241 and the second sensor 243 according to the received signals, the controller 110 may send a message (e.g., through the operation panel 160) indicating which sensor is correctly located (e.g., detects the pulse wave) or indicating which sensor is not correctly located (e.g., does not detect the pulse wave). As such, the user may adjust the position of the sensor module 20 based on the message. For example, in a case that the message indicates that the first sensor 241 is correctly located (e.g., detects the pulse wave) and the second sensor 243 is not correctly located (does not detect the pulse wave), the user may adjust the position of the second end 213 of the rack 210 of the sensor module 20 until the message indicates that both the first sensor 241 and the second sensor 243 are correctly located.

In some implementations, more stringent conditions may be adopted by the controller 110 to determine whether the pulse wave is detected. For example, the controller 110 may determine the pulse wave is detected only when the pulse wave is regular within a preset time period. Specifically, in action S720, except for comparing the threshold and the amplitude of signals from the sensors to find the pulse wave, the controller 110 may further determine whether the pulse wave found is regular within the preset time period. In a case that the controller 110 determines that the pulse wave is regular within the preset time period according to a signal from a sensor, the controller 110 may determine that the pulse wave is detected by the sensor; in a case that the controller 110 determines that the pulse wave is not regular within the preset time period according to a signal from a sensor, the controller 110 may determine that the pulse wave is not detected by the sensor.

For example, according to the found pulse wave, the controller 110 may calculate a standard deviation of the heart rate at several sample time points within the preset time period. In a case that the standard deviation is not greater than a preset threshold, the controller 110 may determine that the pulse wave is regular. However, it is noted that the determination of regular is not limited in the present disclosure. One of ordinary skill in the art may implement the determination as needed.

According to the above, the present disclosure introduces implementations of a physiological signal measurement device in which stiffnesses of two ends of the rack are different and, as such, deformations on the two ends of the rack may be different under a same pressure. Hence, two sensors disposed on the rack can be pressed into a user's skin at different depths when the user is wearing the physiological signal measurement device on his/her limb. In such implementations, a shape of the limbs (e.g., usually in a cone-shape or tapered) and a depth of an artery are taken into consideration such that the signal absorption by tissues (e.g., muscles, fats, etc.) of the user may be eliminated or compensated for. Accordingly, the accuracy of the physiological signal measurement device may be improved.

From the present disclosure, various techniques may be used for implementing the concepts described in the present application without departing from the scope of those concepts. Moreover, while the concepts have been described with specific reference to certain implementations, a person of ordinary skill in the art would recognize that changes may be made in form and detail without departing from the scope of those concepts. As such, the described implementations are to be considered in all respects as illustrative and not restrictive. It should also be understood that the present disclosure is not limited to the particular implementations described above. Still, many rearrangements, modifications, and substitutions are possible without departing from the scope of the present disclosure.

What is claimed is:

1. A physiological signal measurement device, comprising:
a fixing element configured to be fixed on a limb of a user;
a rack configured to engage the fixing element, the rack comprising a first end and a second end distal to the first end;
a first sensor disposed on the first end of the rack; and
a second sensor disposed on the second end of the rack,
wherein the first end has a first stiffness, the second end has a second stiffness, and the first stiffness is higher than the second stiffness.

2. The physiological signal measurement device of claim 1, wherein each of the first sensor and the second sensor comprises an acoustic wave sensor.

3. The physiological signal measurement device of claim 1, wherein the first sensor is configured to detect a first vessel-related signal of the user at a first point on the limb, the second sensor is configured to detect a second vessel-related signal of the user at a second point on the limb, and the first point is proximal to the second point.

4. The physiological signal measurement device of claim 1, wherein:
the fixing element comprises a band strap, and
when the band strap forms a ring in a wearing state, a first perimeter of the ring is longer than a second perimeter of the ring.

5. The physiological signal measurement device of claim 4, wherein the first perimeter of the ring is on a same side as the first end of the rack, and the second perimeter of the ring is on a same side as the second end of the rack.

6. The physiological signal measurement device of claim 4, wherein the band strap has an are shape.

7. The physiological signal measurement device of claim 1, wherein the fixing element comprises a frame part and a fixing part, the frame part is configured to detachably engage the rack, and the fixing part is configured to secure the frame part to the limb of the user.

8. The physiological signal measurement device of claim 7, wherein the frame part of the fixing element comprises a through-hole structure, and the through-hole structure corresponds to the first sensor and the second sensor when the rack engages the frame part of the fixing element.

9. The physiological signal measurement device of claim 7, wherein a thickness of the frame part gradually diminishes from a through-hole side to a fixing part side.

10. The physiological signal measurement device of claim 1, further comprising:
a host module comprising a controller and a first connection interface coupled to the controller,
wherein the rack further comprises a second connection interface coupled to the first sensor and the second sensor, and the second connection interface is configured to connect to the first connection interface.

11. The physiological signal measurement device of claim 10, wherein the host module further comprises:
a housing configured to accommodate the controller and comprising a latch, wherein the latch is configured to detachably engage the rack.

12. The physiological signal measurement device of claim 1, further comprising:
a vibration collection structure corresponding to one of the first sensor or the second sensor,
wherein the vibration collection structure comprises a rigid part and a rubber part covering the rigid part, the corresponding one of the first sensor or the second sensor is located at a center of the rigid part, and the rubber part is configured to contact skin of the user.

13. The physiological signal measurement device of claim 12, wherein the rigid part comprises a cone-shaped structure configured to collect mechanical waves detectable by the corresponding one of the first sensor or the second sensor.

14. The physiological signal measurement device of claim 12, wherein:
the rubber part comprises an innermost portion, at least one middle portion, and an outermost portion, and
the innermost portion and the outermost portion are in contact with the rigid part and the at least one middle portion is not in contact with the rigid part.

15. The physiological signal measurement device of claim 12, wherein:
the rubber part comprises an innermost portion, at least one middle portion, and an outermost portion,
a thickness of the innermost portion and a thickness of the outermost portion are thicker than a thickness of the at least one middle portion, and
the at least one middle portion forms a cavity within the rubber part.

16. The physiological signal measurement device of claim 15, wherein a largest radius of the cavity is not larger than a largest radius of a cone-shaped structure of the rigid part.

17. The physiological signal measurement device of claim 1, further comprises:
a third sensor disposed on the rack and positioned between the first sensor and the second sensor, the third sensor being configured to detect a vessel-related signal of the user, the first sensor, the second sensor, and the third sensor being arranged in a straight line on the rack; and
a controller coupled to the first sensor, the second sensor, and the third sensor, the controller being configured to:
determine whether a pulse wave is detected by both the first sensor and the second sensor; and
in response to determining that the pulse wave is detected by both the first sensor and the second sensor, actuate the third sensor or start to apply the vessel-related signal detected by the third sensor for calculating an output of the physiological signal measurement device.

18. The physiological signal measurement device of claim 17, wherein each of the first sensor and the second sensor comprises an acoustic wave sensor and the third sensor comprises an optical sensor.

19. A physiological signal measurement device, comprising:
a rack comprising a first end and a second end distal to the first end, the first end configured to receive a first sensor and the second end configured to receive a second sensor,
wherein the first end of the rack comprises a first U-shaped recessed structure, the second end of the rack comprises a second U-shaped recessed structure, and two parallel sides of the first U-shaped recessed structure are shorter than two parallel sides of the second U-shaped recessed structure.

20. A physiological signal measurement device, comprising:
a rack comprising a first end and a second end distal to the first end, the first end configured to receive a first sensor and the second end configured to receive a second sensor,
wherein the first end of the rack comprises a first spring, the second end of the rack comprises a second spring, and a spring constant of the first spring is larger than a spring constant of the second spring.
